(19) United States
(12) Patent Application Publication
(10) Pub. No.: US 2018/0098862 A1
(43) Pub. Date: Apr. 12, 2018

(54) INJECTION MOLDED PROSTHETIC LIMB SYSTEM AND RELATED METHODS

(71) Applicant: Rehabilitation Institute of Chicago d/b/a Shirley Ryan AbilityLab, Chicago, IL (US)

(72) Inventors: Todd Kuiken, Oak Park, IL (US); Frank Ursetta, Chicago, IL (US)

(21) Appl. No.: 15/729,063

(22) Filed: Oct. 10, 2017

Related U.S. Application Data
(60) Provisional application No. 62/405,567, filed on Oct. 7, 2016.

(51) Int. Cl. A61F 2/58; A61F 2/68

(52) U.S. Cl. CPC A61F 2/586; A61F 2002/5072; A61F 2/68

(57) ABSTRACT

Injection molded prosthetic limb systems and related methods are disclosed. Example prosthetic hands include a plurality of segments, wherein each segment is comprised of an injection molded plastic.

### RELATED APPLICATIONS

This patent claims priority to U.S. Provisional Patent Application Ser. No. 62/405,567, filed Oct. 7, 2016, entitled “Injection Molded Prosthetic Limb System and Related Methods.” The entirety of U.S. Provisional Patent Application Ser. No. 62/405,567 is incorporated herein by reference.

### FEDERALLY SPONSORED RESEARCH OR DEVELOPMENT

This invention was made with government support under Award No. W81XWH-14-C-0128 awarded by the United States Department of the Army, United States Army Medical Research Acquisition Activity (USAMRAA). The government has certain rights in the invention.

### FIELD OF THE DISCLOSURE

This disclosure relates generally to prosthetics and, more particularly, to injection molded prosthetic limb systems and related methods.

### BACKGROUND

An estimated 41,000 people in the United States live with upper limb loss at or above the wrist level, including more than 350 service men and women with major upper limb amputations attributable to military operations in Iraq and Afghanistan. Estimates of upper limb loss on a world-wide scale are difficult to find, but are likely in the millions. In developing countries, amputation is more common because of poorly regulated industry, less safe transportation, limited access to adequate medical care for infection or injury, and far less complex limb salvage. Adding to this total is violence that includes the deliberate maiming of people, and conflict around the globe.

Loss of an arm causes a major disability that profoundly limits everyday activities such as dressing, eating, and personal hygiene; impacts social interactions, personal relationships, and mental health; and can threaten basic independence. Difficulty in grasping and holding objects impedes leisure activities and, crucially, may severely limit employment opportunities—impacting financial security, sense of identity and purpose, and significantly affecting quality of life. The profound economic, social, and psychological effects of arm loss are especially pronounced in developing countries, where prosthetic technologies and training are poor or non-existent. In the United States, it is estimated that approximately 80% of patients with an upper limb amputation use a prosthesis; however, prosthetic usage in many developing countries is far lower, reflecting the high cost of even simple devices relative to income levels, together with a lack of access to clinicians and resources.

In the prior art, functional upper limb prostheses fall into one of three categories: (1) motorized devices, (2) body-powered devices, or (3) hybrid devices. Motorized devices are powered by batteries and are typically controlled via skin surface electromyographic (EMG) signals that are generated by contraction of residual muscles and recorded by electrodes embedded in the prosthetic socket. Generally, one or two electrodes are placed on opposing residual muscles. When two muscles are available to control one DOF, it is possible to achieve proportional control—in which the speed of movement is proportional to the amplitude of the EMG signals. Additional prosthesis movements may be controlled using various switches and sensors built into the socket.

Body-powered prostheses are controlled by movements of the user's body, such as bi-scapular abduction or glenohumeral flexion. These movements are transmitted through a pulley/Bowden cable system that operates a single degree of freedom (DOF) in the device. For high-level amputees, a switch can be used to change which DOF this movement controls. Many people in the US prefer this type of prosthesis because they are robust, intuitive to use, and relatively inexpensive, and users experience some sensory feedback from the prosthesis through the cables. This type of prosthesis is also commonly used in developing countries—the International Committee for the Red Cross provides components and guidelines for body-powered prostheses for individuals with transhumeral level and transradial level amputations, as these devices are easy to repair and maintain, compatible with climates in different countries, and are low-cost, while providing acceptable function. Body-powered and motorized devices can be combined with a ‘hybrid’ approach; usually the hand is motorized and the proximal components including a wrist and/or an elbow are body powered.

The terminal device of a prosthesis is an important means by which a user interacts with and manipulates their environment, and thus it plays a significant role in determining the user's overall ability to perform the activities that contribute to an acceptable quality of life. Currently, upper-limb prosthesis users in the US may choose from several types of terminal devices: Passive terminal devices are very lightweight, can be cosmetically appealing, and are relatively inexpensive. Although they provide useful passive support and the option of spring-loaded hands, they provide very limited function. Function-specific devices can be either passive or body-powered and are designed for a specific activity, such as lifting weights, playing golf, or discharging a firearm.

Powered, motorized devices include both single DOF hands and multiple DOF hands. Single DOF hands, such as the System Electric Hand (Ottobock), use a single motor and generally consist of two solid fingers that oppose a solid thumb with only a single hinge at the base. These devices allow users to achieve a chuck grasp and are functionally similar to a body-powered hook; cosmetic covers are available. Currently available motorized hands can apply high pinch force, but are very expensive, heavy, and not robust.

Multiple-DOF powered prostheses, such as Bebionic Hand (SteeperUSA) use several motors, which, together with the necessary additional batteries and electronics, increase both the price and weight of the device. For example the Touch Bionics' i-limb ultra Revolution hand, medium size, weighs 451-515 grams, depending on the wrist component, while a medium size Bebionic hand (SteeperUSA) weighs 544-589 grams. To date, the reliability of these complex devices can be poor, and repair generally requires shipment of the device back to the manufacturer for repairs, causing the patient to be without their hand for several weeks at a time. Despite their ability to provide many different grasps, these devices provide less grip force than single DOF devices, the multiple motors can be noisy, and control of these devices can be challenging. The advantages of controlling individual fingers can outweigh the additional weight and noise of the device. Powered devices are generally not available to users in developing countries.

Body-powered devices include prehensors (or hooks) and hands that are opened or closed via a Bowden cable system. Current body-powered hands provide only a 3-jaw chuck grasp, are inefficient, and are hard to use functionally. Furthermore, current body powered hands do not look very natural; they are thick and have only MCP joint articulation. In general, body-powered prehensors (split-hooks and other non-anthropomorphically shaped terminal devices) are considered more functional than body-powered hands by many, although most amputees in the US would prefer an anthropomorphic hand. Globally, the bias is also toward hands over hooks, as many cultures are unwilling to accept the appearance of a prosthetic hook, and individuals with arm amputations in these cultures frequently choose to not to wear a prosthesis instead of using a hook terminal device.

There are two general types of body-powered terminal devices. With voluntary-opening (VO) devices, the user exerts force on the cable to open the device but can then relax and allow the prehensor to grasp an object; rubber bands or springs supply the force needed to close the device. VO devices are easy to use, but the rubber bands or springs provide a weak grasp force of only a few pounds. Achieving a higher grip force means that the user must overcome stronger spring forces to open the device, even to manipulate lightweight objects, and the user is limited to the grip force provided by the spring. Voluntary-closing (VC) devices require the user to pull on the cable to close the device. VC devices allow the user to control grip force—from large pinch forces, as large as the user can generate—to very low forces for more delicate objects. However, continual user-generated force is required to maintain any pinch force; if the user relaxes they will drop any object they are holding unless a locking mechanism is used.

Advances in prosthetic technology and control systems continue to provide better robotic options for individuals with arm loss. Unfortunately, these devices are frequently not covered by insurance. The cost of such robotic devices is often prohibitive, even for individuals in the US with medical insurance. The Medicare allowable cost (Centers for Medicare and Medicaid services 2015 region B fee schedule) for even a basic single degree of freedom (DOF) powered hand is approximately $6,500. The newer multifunction prosthetic hands would cost over $33,000. Body-powered hands are much more affordable with a Medicare reimbursement rate (Centers for Medicare and Medicaid services 2015 region B fee schedule) of $939.60 for a VO BP hand and $1333.53 for a VC hand. However they hold only a small part of the market due to their limitations and poor cosmesis. The high costs of current prosthetic devices put all of these devices beyond the financial resources of the vast majority of people in the world.

The less expensive, but not inexpensive, single DOF body-powered and motorized hands look very bulky even with gloves on, and the rigid fingers look unnatural. Although the more complex multifunction myoelectric hands have thinner palms and fingers that bend at the anatomic equivalent of the metacarpophalangeal (MCP) joint and the proximal interphalangeal joint (PIP), they all require a glove for cosmesis, and these gloves impair performance. Though powered prosthetic hands offer several advantages over body-powered devices—they do not require a harness and cable, resulting in a more anthropomorphic appearance, and do not require as much strength and range of motion of the torso—they remain less popular than body-powered hooks as they are expensive, heavy, and less robust. Clearly, some important gaps still remain in the type of prosthetic hands currently available.

Underactuation is a method used to reduce the number of actuators without reducing the number of DOFs. This approach allows adaptive grasping, i.e., the hand can conform to and grasp different shapes, much like an intact hand. Using fewer actuators reduces device weight and complexity. Many underactuated prosthetic hands have been developed using a variety of mechanism to power the digits. To date, the dimensions and weight of these hands have been too large for use by an average person. Robotic underactuated hands have been developed, but size, shape, and weight constraints make applying these design concepts to prosthetic hands difficult, and few underactuated hands have become clinically relevant. Underactuation has generally been achieved using rigid linkage mechanisms, which allow for large forces but add weight and require thick fingers that would reduce cosmesis in a prosthetic hand. Cable-driven mechanisms allow for a potentially lighter design and reduced finger size. One popular mode of underactuated finger flexion is to use a whippletree mechanism, in which cables are threaded through each digit and then attached to cross bars located in the palm. Pulling on the cable flexes the digits to create a grasp. One prior art effort involved development of a hand using the whippletree concept in which one ultrasonic motor pulled the cables and cross bars proximally to flex the fingers and thumb. A disadvantage of this method has been that space is required in the palm for the cables and bars to be pulled into, which does not leave ample room for a motor or wrist actuator.

3-dimensional (3D) printing is a new design tool that is evolving quickly. However, 3D-printed components are not strong enough for a prosthesis, except perhaps for limited use by young children. The plastics do not have a high strength rating (especially for hinges) and their strength further degrades over the course of weeks to months. Furthermore, 3D printed parts are not inexpensive unless one already has access to a quality 3D printer.

### BRIEF DESCRIPTION OF THE DRAWINGS

FIG. 1A illustrates an example powered hand prosthesis including flexion cables attached to a spool that is driven by a motor, in accordance with aspects of this disclosure.

FIG. 1B illustrates an example body-powered hand prosthesis including flexion cables attached to a spool that is driven by a an actuation (Bowden) cable.

FIG. 2 shows the example hand prosthesis of either of FIG. 1A of FIG. 1B having an adaptive grasp of a plastic toy.

FIG. 3 illustrates an example spool that may be used to implement the hand prostheses of FIGS. 1A and 1B.

FIG. 4 illustrates an example body-powered hand prosthesis having a spool with an enlarged diameter where a Bowden cable is attached to the spool, to increase the pulling force of the Bowden cable on the spool.

FIG. 5 illustrates the example hand prosthesis of any of FIG. 1A, 1B, or 4, executing a pinch grasp.

FIG. 6 displays a distal structure of an example finger for any of the example hand prostheses disclosed herein, with a fused, flexed distal interphalangeal joint at 20 degrees.

FIG. 7 displays a cross-section of an example finger for any of the example hand prostheses disclosed herein, including a flexion cable routing through a spring and a flat spring at the rear of the finger.

FIG. 8 displays another example individual finger, showing steel joints at the metacarpophalangeal and proximal interphalangeal locations.

The figures are not necessarily to scale. Where appropriate, similar or identical reference numbers are used to refer to similar or identical components.

### DETAILED DESCRIPTION

Upper-limb amputees need prostheses that can effectively replace lost function, enabling them to return to work and participate actively in society. As detailed below, we have developed a hand with a novel and innovative under-actuated mechanism that can be used with either body-powered or motorized actuation. This hand is robust, has good cosmesis without a glove, and enables compliant, adaptive grasps. The hand may be manufactured using molds so that stronger injection-molded plastics can be used for the final product, enabling inexpensive large-scale manufacture of a robust hand at low cost. This hand is more aesthetically pleasing and more functional than body-powered hand options currently available to US consumers, and its low cost makes it potentially accessible to users around the world.

There is an urgent need for robust, functional, and inexpensive upper limb prosthetic hands that look more natural, with a thin palm, a wrap-around grasp, and articulation of the finger MCP and PIP joints. These devices should be robust, lightweight, and ideally simple enough serviced or repaired without having to go back to the manufacturer. Finally, as highlighted above, they need to be much less expensive than current options to meet the both needs of individuals in the US and the much larger global population. These hands would also be transformational for individuals in developing countries whose prosthetic options are even more severely limited by cost and access to technology and clinical care.

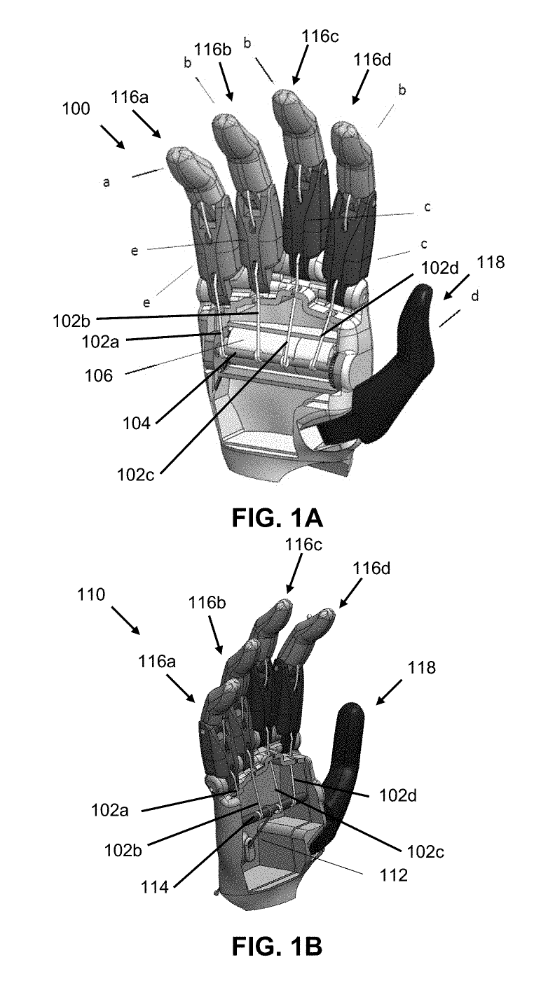

We have developed a hand, embodiments of which are shown in FIGS. 1A and 1B with a novel and highly innovative underactuated mechanism that can be used with either body-powered or motorized actuation. This hand is robust, has good cosmesis without a glove, enables compliant, adaptive grasps, and can be manufactured inexpensively in large quantities using low-cost materials and production methods.

Our hand design can use either body-powered or motorized actuation. FIG. 1A displays a powered hand 100 showing flexion cables 102a-102d attached to spool 104 that is driven by a motor 106. FIG. 1B displays a body-powered hand design 110 showing an actuation (Bowden) cable 112 attached to a spool 114. In both example hands 100, 110, the palm cover has been removed and is not shown. Each of the example hands 100, 110 includes fingers 116a-116d and a thumb 118. Replication may be taken advantage of where possible to reduce the number of unique parts. In the embodiment shown in FIG. 1A, the hand is made up of unique plastic parts (identical parts are indicated by identical letters in FIG. 1A); some parts are reused for multiple hand components, for example, the distal links (b) for the index, middle and ring fingers may all use the same part. This can be done where it poses no mechanical drawback and anthropomorphic appearance is not compromised.

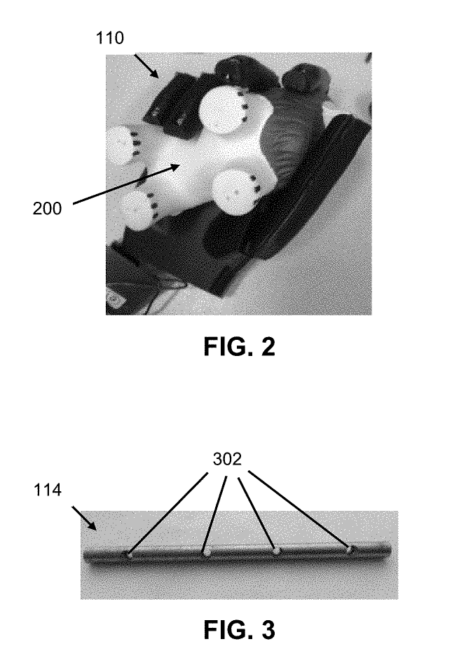

The fingers 116a-116d are driven by the flexion cables 102a-102d (similar to the flexion tendons in the human hand) and bend through an anatomic range of motion at the MCP and PIP joints. One aspect of the example prosthetic hands 100, 110 is that that these flexion cables 102a-102d wrap around a single, space-efficient spool 104, 114 in the palm of the hand 100, 110. Force exerted through the Bowden cable 112 turns the spool to flex the fingers to provide a forceful flexion grasp. For instance, in FIG. 2, the hand 110 is shown having an adaptive grasp of an awkwardly shaped plastic toy 200. A motorized hand would work in essentially the same manner, with a single motor drive spool rotation. Extension is provided with a contoured leaf-spring on the back of each finger that defines how the PIP and MCP flex with a given actuation force. This allows for natural-appearing and consistent movement of the fingers. Compliance is provided by springs in series with the cables of the 4^{th} and 5^{th} digits 112a, 116b (e.g., ring and little fingers), which enables them to conform around the object being grasped—both a functional and aesthetic feature. The 2^{nd} and 3^{rd} digits 116c, 116d (e.g., index and middle fingers) are not compliant, enabling an effective 3 jaw chuck to be achieved. The thumb 118 is manually rotated by the user and opens fully for a flat palm posture. When the thumb 118 is positioned opposite the index finger 116d, the hand 110 can close in a form of power grasp around an object, with the 4^{th} and 5^{th} digits 116a, 116b conforming to the object′ shape (see FIG. 2). The thumb 118 can be aligned to oppose the index finger 116d to achieve a fine pinch grasp, or placed in its most adducted position to achieve a strong and reliable 3-jaw chuck grasp.

The hands 100, 110 may be comprised of injection molded plastic components together with metal hinges. Molds are relatively expensive to build; however, once the molds are made, the parts become very inexpensive to manufacture, and the plastic is much stronger. Also the color of a batch of parts can easily be changed, thus the hands 100, 110 can be made in any color to match a large variety of skin tones. Injection-molded plastic can be made very strong with certain techniques, such as incorporating carbon fiber strands in the plastic. The plastic is easily cleaned (compared to rubber or silicone gloves). Since the color of the part is not just surface paint, the color does not scratch off, and any scratches or marks that come with use can be sanded or buffed out, making these hands very durable. In various embodiments, the hand may include additional cosmetic details, such as finger nails and some skin lines to enhance realism, thus no gloves are needed (reducing costs and weight further). Another attribute in the simplicity of the design is that the hand should be relatively easy to service by an ordinary person with some skill in tools and repair (perhaps using an instructional video on the internet such as a YouTube video). If a cable 102a-102d breaks, a new one can be installed; if a finger 116a-116d, 118 breaks, a new finger can be installed. Finger joints are common failure points in prosthetic hands. In a preferred embodiment, stainless steel joints that clip into the plastic fingers may be used to connect segments of the fingers 116a-116d. A hand may also use exact copies of a single metal joint design for all MCP and PIP joints on all fingers, which reduces cost and simplifies the design to facilitate repair.

The example prosthetic hands can be very light. Even with its additional functions of a wrap-around grasp and moveable thumb, an embodiment weighs about the same as other body-powered hands. In an embodiment, the body-powered hand weighs approximately 321 g with a standard thread bolt for coupling.

The example hands 100, 11 may be voluntary opening (VO) or voluntary closing (VC). A VC hand has a cosmetically appealing resting state in what appears to be a relaxed hand open position—thus it may have a preferred cosmesis compared to a VO hand that has a default closed position. Also an open hand is still a very useful posture for the user, as it can be used to hold or support objects.

The hand 110 may have cable-actuated fingers 116a-116d, all driven by a single Bowden cable 112. The hand may comprise a spool 114, as shown in FIG. 3. The example spool 114 has through holes 302, such as the four holes shown in FIG. 3, to attach the flexion cables 102a-102d. The spool 114 may be turned by user-generated force through the Bowden cable 112, winding the flexion cables 102a-102d around the spool 114 to flex the fingers 116a-116d.

The thumb may be manually positioned and locked into predefined orientations to facilitate up to three separate grasps. The size and shape of the hand may be modeled on the dimensions for the 50^{th} percentile male, because over 70% of upper limb amputees are male, both in the US and in other countries. A single finger can exert 10 N of force, which is adequate for most activities of daily living. Different size hands can be obtained by varying finger length and palm size. Creating two additional palm sizes, for example, based on the 25^{th} percentile male and the 25^{th} percentile female would be reasonable and would cover most of the US amputee population. A variety of finger and palm sizes would enable the building of hands that fit a very wide range of body sizes. Certain fingers could be used for multiple size hands, reducing the overall the number of plastic parts needed for different sized hands, thus three more distal structures and three more proximal structures should result in enough finger combinations to make the two additional hand sizes. Different thumb sizes may be used for each hand size.

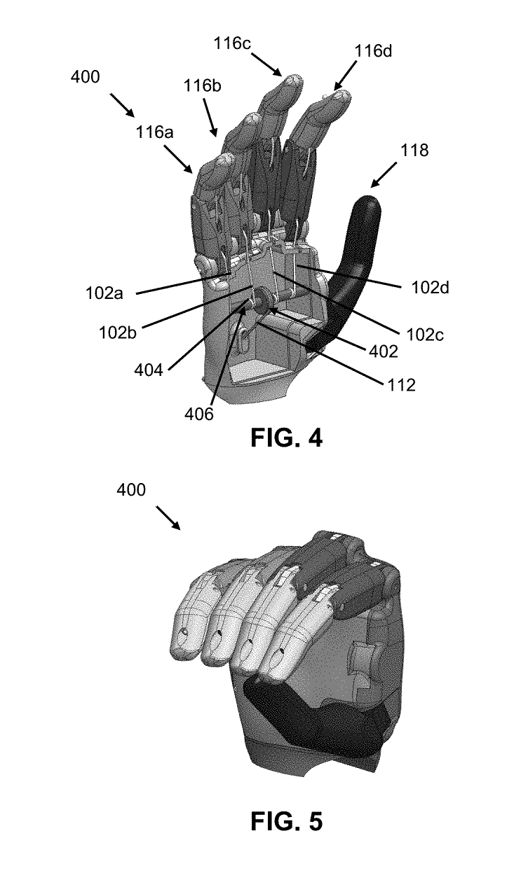

The spool-driven design allows force adjustments to be made by increasing the diameter of the spool 114 where the Bowden cable attaches. FIG. 4 shows a display of a spool-driven prosthetic hand 400, with an enlarged diameter 402 where the Bowden cable 112 is attached to the spool 404 in order to increase the pulling force of the cable 112 on the spool 404, and a smaller diameter 406 in other locations whether the flexion cables 102a-102d are attached to the spool 404. This will allow the user additional grip force if desired, and enable simple modification of the hand design to meet different user needs.

Power and fine pinch grasps are achievable through a manually positioned thumb 118. A 3-jaw chuck grasp is also shown, at FIG. 5.

Finger flexion can be driven by the flexion cables 102a-102d attached to each individual finger end. The cables 102a-102d are routed through the fingers 116a-116d such that, when the cable 102a is tensioned, the corresponding finger 116a flexes inward towards the palm of the hand 100, 110, 400. Both the proximal interphalangeal (PIP) joints and the metacarpophalangeal (MCP) joints bend. The cable tension is driven by a spool 114, such as the spool shown at FIG. 3 with the through holes 302 to allow attachment of all of flexion cables 102a-102d. The spool 114 is rotated as the user pulls on the Bowden cable 112, winding the flexion cables 102a-102d around the spool 114.

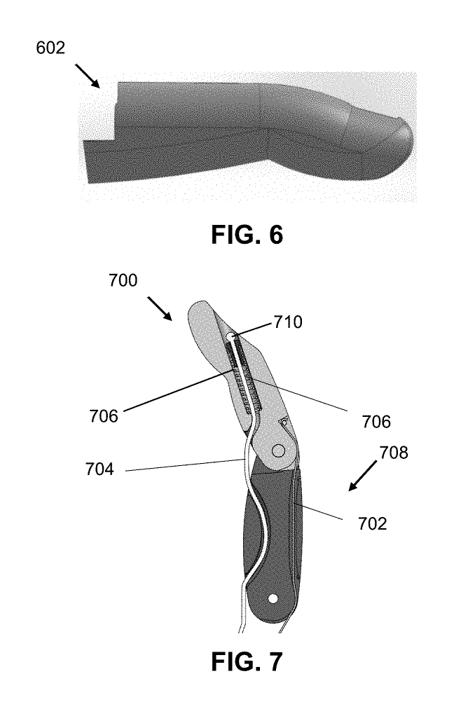

The distal interphalangeal joint (DIP) of each finger 116-116d may have a fixed angle of flexion, as shown in FIG. 6. For instance, the fixed angle of flexion may be 20 degrees. Fingers can be set in the frontal plane of the hand and may be splayed for aesthetic reasons. FIG. 6 displays a distal structure of a finger 602 with a fused, flexed DIP at 20 degrees.

Extending the fingers back to the straight position may be achieved using springs. Flat springs 702 can be seen in FIG. 7, which also displays a cross-section of an individual finger 700 showing the flexion cable 704 routing through a distal spring 706 and the flat spring 702 at a rear side 708 of the finger 700. The flat spring 708 can be inserted into the backhand side of each of the finger 700. The flat spring 70 are bent as the fingers are flexed inward. When the tension in the flexion cable is released, the springs regain their shape and straighten the finger joints. The springs are pre-tensioned such that the MCP joint flexes completely before the PIP joint flexes. This allows for a secure three jaw chuck grip (shown in FIG. 5) and ensures that a wide grasp can be achieved.

Compliance in each finger may be useful, in order to create a stable hand grasp. This is because the cables that drive the fingers to flex all rotate about the spool equally. Without compliance, when one finger contacts a solid object and stops, the others immediately stop as well. The result would be that only one finger really grasps the object. Including compliance allows the fingers to flex further and allows multiple fingers to grasp an object. Individual finger compliance is achieved by inserting the distal spring 706 into the ends of each of the fingers 700. The flexion cables 704 are each attached to a ferrule 710 at the end of the fingers. As the flexion cables 704 are tensioned and a finger 700 grasps a rigid object, the ferrule 710 compresses the distal spring 706, allowing the cables 704 to be further wound on the spool 104, 114, 404. Final grasp position occurs when one of the distal springs 706 has compressed fully and the cable 704 can no longer be wound by the actuation cable 112. The routing of the flexion cable 704 through the distal spring 706 and ferrule 710 can be seen in FIG. 7.

Maintaining a grasp requires continuous force exertion in a VC device, which can be tiring or inconvenient. A locking mechanism can be added so that once an object is grasped, the user can lock the device and no longer needs to exert force to maintain the grasp. The grasp can be released by unlocking the mechanism.

The thumb 118 will be manually positioned and locked into predefined orientations by the user. A simple notch and ball-nosed plunger may be used to hold the thumb 118 securely in position. The thumb is hinged such that the force acting upon the thumb tip is perpendicular to the axis of rotation, thus resisting rotation during use.

Reliability

The flexion cables will see a variety of loads and will be sliding across plastic surfaces. Therefore it is important that a very resilient cable be chosen. Spectra 400 Ultra can be used for its high tensile strength (400 lbs or approximately 1760N), its abrasion resistance, and its low coefficient of friction. It is fifteen times stronger than steel by weight and does not corrode. The individual flexion cables should see no more than 160N of force, which is well below the tensile strength of the Spectra cable. Teflon liners can be used to mitigate any impact of abrasion or friction.

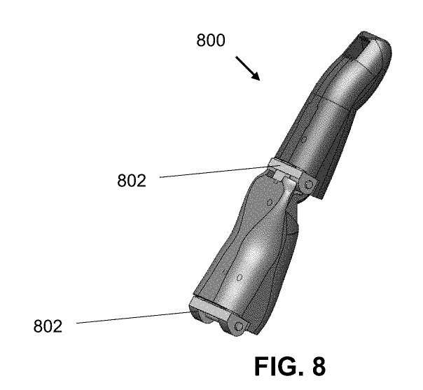

FIG. 8 displays an individual finger 800, showing steel joints 802 at the MCP and PIP locations. The finger joints in this design will see moderate loads with relatively small rotation angles and speeds. The joints 802 may be machined into high strength plastic fingers and hand. An additional low cost option is to use steel bushings that can be inserted into the joint and make it more resilient. These inserts can be extended as far as needed into the finger parts for added strength. The hinge pins can be removable so that fingers can easily be removed and replaced if necessary. The steel inserts can be pressed into the finger parts for quick and reliable manufacturing. The same steel hinge on all finger joints 802 may be used, to keep the hand simple and low cost.

Resistance to backhand impact can be achieved through compliance. Many prosthetic hand designs use linkages to flex individual fingers. Those designs are susceptible to incidental impacts from the backhand side. During such an impact, the rigidity of finger linkages puts a high load on hinges and may cause damage. The compliance of the cable-driven finger design allows for resilience against backhand side impacts, as the fingers would merely close into the palm without damaging any of the components. The springs then straighten the fingers out once the force is removed.

The flat springs that straighten the fingers will see many load cycles. High strength, constant force, stainless steel springs can be used to resist wear. They are rated for 4,000 cycles, but if these are determined to be insufficient, higher cycle-life springs are available.

The compression springs that are used to allow for compliance in the individual fingers will only be exposed to compressive forces and should not yield.

The material that will make up the vast majority of the prosthetic hand will be a high strength plastic, which, generally speaking, is a low cost material. Injection molding allows for more diverse material options at a low per-part cost. Glass-filled nylon, for example, is a high strength option that can be manufactured in an injection mold.

Using injection molded plastics for manufacturing will allow us to have the higher quality plastic at a cost that is even lower than the original in house printed parts. We estimate that plastic fabrication method would be below $100. Further cost savings can be found in purchasing the other parts in high quantities for an expected cost per hand of below $100, for example the metal hinges. Once the initial cost of the injection molds has been met, the individual part cost will be extremely low. Either aluminum or steel molds may be employed.

Cosmetic gloves contribute significantly to the overall weight of prostheses, and can reduce performance, so disclosed example prosthetic hands may be designed for use without a glove.

While the present method and/or system has been described with reference to certain implementations, it will be understood by those skilled in the art that various changes may be made and equivalents may be substituted without departing from the scope of the present disclosure. In addition, many modifications may be made to adapt a particular situation or material to the teachings of the present disclosure without departing from its scope. For example, systems, blocks, and/or other components of disclosed examples may be combined, divided, re-arranged, and/or otherwise modified. Therefore, the present method and/or system are not limited to the particular implementations disclosed. Instead, the present methods, apparatus, and/or systems include all implementations falling within the scope of the appended claims, both literally and under the doctrine of equivalents.

What is claimed is:

1. A prosthetic hand comprising a plurality of segments, wherein each segment is comprised of an injection molded plastic.

2. The prosthetic hand as defined in claim 1, comprising a spool connected to one or more of the segments via corresponding flexion cables in the one or more segments.

3. The prosthetic hand as defined in claim 2, wherein the spool is configured to rotate in a first direction to flex the segments and to rotate in a second direction to release the segments.

4. The prosthetic hand as defined in claim 2, wherein at least one of the flexion cables is routed through at least one of the segments of a finger and is coupled to a first spring at a distal end of the one of the finger.

5. The prosthetic hand as defined in claim 4, wherein the spring and the one of the flexion cables are configured to provide compliance of the corresponding member.

6. The prosthetic hand as defined in claim 4, wherein at least one of the segments of the finger comprises a second spring biased to extend the finger.

7. The prosthetic hand as defined in claim 2, further comprising a motor configured to actuate the spool.

8. The prosthetic hand as defined in claim 2, further comprising a Bowden cable configured to actuate the spool.

9. The prosthetic hand as defined in claim 7, wherein the spool has a first diameter where the flexion cables are coupled to the spool and a second diameter where the Bowden cable is coupled to the spool.

10. The prosthetic hand as defined in claim 1, wherein at least one of the segments is a thumb finger that is manually positionable.

11. The prosthetic hand as defined in claim 1, further comprising metallic joints configured to pivotally couple two or more of the segments corresponding to a same finger.

12. The prosthetic hand as defined in claim 1, wherein the prosthetic hand is voluntary opening or voluntary closing.
